(19) United States
(12) Patent Application Publication
(10) Pub. No.: US 2021/0122635 A1
(43) Pub. Date: Apr. 29, 2021

(54) METHOD OF CONSTRUCTS FABRICATION FROM CALCIUM PHOSPHATES

(71) Applicant: VIVAX BIO, LLC, New York, NY (US)

(72) Inventors: Vladislav Aleksandrovich PARFENOV, Podolsk (RU); Yusef Dzhorzhevich KHESUANI, Moscow (RU); Vladimir Aleksandrovich MIRONOV, Moscow (RU); Elena Anatolievna BULANOVA, Moscow (RU); Elizaveta Valerievna KOUDAN, Moscow (RU); Elizaveta Konstantinovna NEZHURINA, Moscow (RU); Pavel Anatolievich KARALKIN, Moscow (RU); Frederico DAS PEREIRA, Vitoria da Conquista (BR); Stanislav Vladimirovich PETROV, Moscow (US); Alexander Yurievich OSTROVSKIY, Moscow (RU); Yakov BALAKHOVSKY, New York, NY (US)

(21) Appl. No.: 17/075,006

(22) Filed: Oct. 20, 2020

Related U.S. Application Data
(60) Provisional application No. 62/923,809, filed on Oct. 21, 2019.

(51) Int. Cl. C01B 25/32; A61L 27/12; B01J 19/08

(52) U.S. Cl. CPC C01B 25/321; A61L 27/12; B01J 2219/089; A61L 2430/02; B01J 2219/0852; B01J 19/087

(57) ABSTRACT

This invention newly describes fast and reliable creation of three-dimensional scaffolds using magnetic levitation of calcium phosphate particles. In particular, tricalcium phosphate particles of similar size and specific porosity are used which are subjected to recrystallization process after magnetic levitation assembly of the scaffold to ensure scaffold cross-linking. Levitation assembly without marks is reached by using specially developed magnetic system in the presence of gadolinium salts which allows levitation of calcium phosphate particles. Chemical synthesis of octacalcium phosphate in magnetic levitation conditions in non-homogeneous magnetic field was also demonstrated. Such approach allows to obtain phosphate phase quickly in biocompatible finished product.

### TECHNICAL FIELD

This group of inventions is related to the method of materials creation for restoration of bone tissue defects in orthopedics, dentistry, traumatology, reconstructive surgery, maxillofacial surgery, and other fields.

### BACKGROUND OF THE INVENTION

Bone is a mineral-organic composite consisting mainly of calcium phosphate (CP) also including proteins (mainly type I collagen) and water. In bone tissue CP is presented in the form of crystallized or partly crystallized hydroxyapatite (HA) [1]. Therefore, application of 3D scaffold based on CP is a promising solution for bone defects restoration. The use of CPs as material for bone transplant has some advantages such as biodegradability, biocompatibility and osteoconductive properties. Most synthetic CP materials used in clinical practice are based on α-tricalcium phosphate (α-TCP) ceramics which is a reliable osteoconductive material commercially available in the form of granules, cements, or blocks [2-4]. However, degradation rate is often lower than osteogenesis rate which leads to accumulation of CP materials in a bone defect area. HA synthesis can be achieved using several methods, but in all cases synthesized phase differs from native HA [5]. For this reason, a biomimetic approach was suggested using octacalcium phosphate (OCP) as HA predecessor in a natural bone tissue [6]. It is worth noting that reproduction of OCP phase in chemical reaction is a very difficult task which usually takes about two weeks [7]. Therefore, development of technology for faster and reliable OCP phase preparation is an important objective for fabrication of scaffolds intended for bone defect restoration. Furthermore, synthetic OCP granules may be considered as building blocks for fast prototyping, i.e. 3D printing of tissue-engineered bone scaffolds [7].

In the past decade several widely used methods were developed for fast prototyping of 3D bone transplant based on ceramics [8-11]. Fast prototyping is an additive technology based on layer-by-layer fabrication of 3D objects including several steps: (i) target object computer model creation; (ii) 3D model slicing; (iii) layer-by-layer fabrication of an object of required size, shape and inner structure using different approaches to 3D printing such as laser stereolithography [12], extrusion printing [7], material jetting [13,14]. Material jetting is the most suitable approach to fabrication of solid scaffolds (frameworks) with predefined geometry. During material jetting process printing head moves in accordance with preset trajectories over the surface where powder is homogeneously distributed. Printing head applies adhesive substance on the powder sticking powder particles together layer by layer which allows fabrication of solid objects in accordance with developed 3D model. Nevertheless, additive approach takes a lot of time and has other limitations such as limited tool resolution and necessity of additional material.

Forming technology which arisen recently as an alternative to additive approach includes assembly in the whole volume of a working chamber. In forming approach, scaffold shape and microstructure are defined by physical forces such as acoustic waves, magnetic and electric fields. Desired geometry of 3D scaffold in accordance with preliminary 3D model is achieved by calculation and modeling of certain physical fields distribution in 3D space. Therefore, magnetic field is most commonly used for levitation and modification of different objects (magnetic levitation (MagLev)). In order to provide conditions for diamagnetic objects levitation for magnetic assembly, paramagnetic medium, for example, with gadolinium salts, may be added. Gadolinium (Gd³⁺) based pharmaceutical preparations usually used by MagLev approach are approved by the FDA for clinical use as contrast agents for MRI and, therefore, contain non-toxic agents. Magnetic levitation assembly was recently suggested as a new fabrication method for biomaterials and scaffolds for tissue engineering [15-18]. In other research magnetic fabrication of viable scaffolds based on chondrospheres using levitation was demonstrated [19].

### DISCLOSURE OF INVENTION

The objective of the present invention is to develop fast and reliable production method of material for restoration of bone tissue defects based on magnetic levitation assembly in non-homogeneous magnetic field allowing generation of biocompatible three-dimensional material based on calcium phosphate for restoration and regeneration of bone defects.

The technical result of the invention is the development of fast and reliable material generation method for restoration of bone tissue defects based on magnetic levitation assembly in non-homogeneous magnetic field allowing generation of biocompatible three-dimensional material based on calcium phosphate for restoration and regeneration of bone defects. This method allows to create 3D scaffolds based on octacalcium phosphate (OCP) quickly, in particular, within 40 hours, in levitation conditions, excluding the use of temporary foundation, caps and magnetic marks. Furthermore, according to SEM (scanning electron microscopy) data and X-ray structural analysis, paramagnetic salts, in particular, gadolinium salts do not remain in the structure and on the surface of CP particles. Furthermore, 3D scaffold generated using the method according to the invention has sufficient porosity level which can provide cellular migration within porous channels. Alternatively, cells or tissue spheroids can be placed in granular space of 3D scaffold. It makes the resulting 3D material a promising candidate for application in tissue-engineered approaches to bone regeneration. Therefore, the method according to the invention offers an opportunity for fast assembly and fabrication of material (or scaffold) based on CP. Design and development in this field can play an important role in the field of bone tissue engineering.

The said technical result is achieved by realization of the method of fabrication of 3D material based on octacalcium phosphate by magnetic levitation assembly in non-homogeneous magnetic field including the following phases:

1) α-tricalcium phosphate particles assembly in 3D structures,

2) recrystallization of obtained structures.

In particular embodiments of the invention magnetic levitation assembly is performed in the central area on non-homogeneous magnetic field with lowest field density parameters from 3D material randomly distributed within fabrication chamber volume.

In particular embodiments of the invention non-homogeneous magnetic field is created using magnetic system consisting of at least two circular neodymium magnets with analogous poles facing each other.

In particular embodiments of the invention non-homogeneous magnetic field is created using Bitter magnets.

In particular embodiments of the invention the size of α-tricalcium phosphate particles is 250 to 500 μm.

In particular embodiments of the invention α-tricalcium phosphate particles assembly is performed by levitation forming method using non-homogeneous magnetic field in paramagnetic medium. In particular embodiments of the invention paramagnetic medium contains Gd³⁺ gadolinium salts.

In particular embodiments of the invention α-tricalcium phosphate particles assembly is performed at temperature of 0 to 80° C. In other embodiments of the invention α-tricalcium phosphate particles assembly is performed at room temperature.

In particular embodiments of the invention α-tricalcium phosphate particles are placed in magnetic field in buffer No. 1 with mass ratio of α-tricalcium phosphate particles to buffer No. 1 equal to 1:100 to 1:400, where buffer No. 1 is an aqueous solution of 1.5±0.1 M of sodium acetate and 1.0±0.01 M of phosphoric acid with pH 5.2±0.2 and also contains 3M of Gd³⁺.

In particular embodiments of the invention mass ratio of α-tricalcium phosphate particles to buffer No. 1 is 1:400.

In particular embodiments of the invention recrystallization of obtained structures is performed in two stages: in the first stage obtained 3D structures are held in magnetic field during 12 to 48 hours, then buffer No. 1 is replaced by buffer No. 2 and held in magnetic field during more 12 to 48 hours, where buffer No. 2 represents an aqueous solution of 1.5±0.1 M of sodium acetate with pH 8.2±0.2 and also contains 3M of Gd³⁺. In more specific embodiments of the invention obtained 3D structures are held in magnetic field during 20 hours in the first stage. In other embodiments of the invention 3D structures are held in magnetic field during 20 hours after buffer No. 1 replacement by buffer No. 2.

In particular embodiments of the invention α-tricalcium phosphate particles are placed in magnetic field in buffer No. 1 in transparent syringe and preliminarily shaken.

The present invention also contains 3D material based on octacalcium phosphate for restoration and regeneration of bone defect obtained using the method according to the invention.

In theory, magnetic levitation approach may be used for assembly of spatial 3D scaffolds from single CP granules. But, in order to compensate for gravitation, CP levitation requires magnetic force greater than the force required for living cells levitation by several fold due to the difference in material density. This problem may be solved by the use of higher magnetic field gradient which may be achieved by the use of superconducting electromagnets (of Bitter electromagnets) or higher concentrations of paramagnetic salts in the medium. The basic concept of the present invention is based on assembly of 3D scaffolds (frameworks) from octacalcium phosphate (OCP) in levitation conditions excluding the use of any temporary foundation, caps, and magnetic marks. Authors of the present invention believe that as a result of realization of the present invention more advanced biodegradable scaffolds may be fabricated with enhanced properties in magnetic levitation conditions. Authors of the present invention newly present successful fabrication of biocompatible framework using forming technology for restoration and regeneration of bone defect, namely, the method of CP particles assembly by means of magnetic levitation using gadolinium salts in special buffer solution with subsequent recrystallization in levitation conditions. This approach allows to create 3D scaffolds based on OCP phases quickly, in particular, within 40 hours.

In additions to recrystallization rate, another important property of scaffold (material) according to the invention is its porosity. Scaffold assembly involves the most dense packing of spherical particles. The space between granules is called interpenetrating pores and there is a linear correlation between interpenetrating porosity with the most dense hexagonal packing of spherical particles and their diameters. Therefore, application of porous particles additionally provides osteoconductive properties and use of ceramics with bimodal distribution of pores by size seems preferable. 3D scaffolds generated using the method according to the invention have sufficient porosity level which can provide stem cells migration within porous channels. Alternatively, cells or tissue spheroids can be placed in granular space of 3D scaffold. This document contains research results obtained on primary adherent cultures mesenchymal stem cells (MSCs) from primary teeth pulp samples collected after normal peeling-off—SHED (stem cells of peeled-off human primary teeth). These cells are naturally involved in physiologic regeneration processes and may be subjected to in vitro osteogenic differentiation. It makes them promising candidates for application in tissue-engineered approaches to bone regeneration. Therefore, the method according to the invention may be used for fabrication of scaffolds for tissue engineering based on OCP and MSC intended for bone defect regeneration and bone tissue restoration.

### DETAILED DESCRIPTION OF THE INVENTION

### Brief Description of Drawings

FIG. 1. α-TCP particles fabrication:

(a) Schematic image of α-TCP particles fabrication process;

(b) SEM image of α-TCP particles (bar 200 μm and 20 μm);

(c) α-TCP particles diameter distribution;

(d) X-ray phase analysis of α-TCP particles.

FIG. 2. Magnetic device:

(a) Schematic image of the device and fabrication of scaffold (framework);

(b) Horizontal section of magnetic field (arrows show magnetic well);

(c) Vertical section of magnetic field;

(d) Dependence of the distance between levitation unit and magnetic field center on the ratio between magnetophoretic and gravitation forces acting on the particles;

(e) Magnetic printer model.

FIG. 3. Levitation assembly of 3D scaffold based on CP in paramagnetic medium using magnetic field (concentration of gadolinium salt is 3 M):

(a) Modeling of α-TCP particles assemble and scaffold shape.

(b) α-TCP particles assemble dynamics.

(c) Assembly time area curves in modeling and in experiment, highlighted in red and blue, respectively.

FIG. 4. Fabrication of 3D scaffold (framework) based on CP in paramagnetic medium using magnetic field (concentration of gadolinium salt is 3 M):

(a) Schematic image of recrystallization phases.

(b) SEM image shows the surface of primary α-TCP particles (bar 10 μm).

(c) SEM image shows dicalcium phosphate dihydrate (DCPD) phase (intermediate product) (bar 10 μm).

(d) SEM image shows the surface of obtained scaffold (OCP phase) (bar 10 μm).

FIG. 5. Biocompatibility of 3D scaffolds and illustration of 3D scaffolds fabrication using magnetoacoustic field:

(a) Impact of medium after 4 days of incubation with 3D scaffold based on CP on cell viability (Alamar Blue analysis, 72 hours).

(b) SHED tissue spheroid made of 27,000 cells in spheroidal microplate (bar 100 μm).

(c) 3D scaffold based on CP immediately after fabrication (bar 2,000 μm).

(d) Schematic image of adherence and migration of SHED cells on the surface and within 3D scaffold.

(e) 3D scaffold based on CP incubated with SHED spheroids (0 hours).

(f) 3D scaffold based on CP incubated with SHED spheroids during 7 days.

(g) Images of fluorescent and light microscopy of 3D scaffold incubated with SHED spheroids during 7 days.

(h) SEM image of 3D scaffold with SHED spheroids on the surface after 7 days of incubation (bar 50, 100 μm).

FIG. 6. Phase analysis of 3D scaffold (framework):

(a) X-ray phase analysis of 3D scaffold fabricated by magnetic levitation after step 1 (20 hours in buffer No. 1);

(b) X-ray phase analysis of 3D scaffold fabricated by magnetic levitation after step 1 (20 hours in buffer No. 1) and step 2 (20 hours in buffer No. 2).

FIG. 7. SEM image of scaffold (framework) fabricated without exposure to magnetic field.

FIG. 8. Phase-contrast microscopy of primary SHED culture:

(a) after the first passage

(b) in the passage.

FIG. 9. SHED multilinear differentiation:

(a) Osteogenic differentiation was detected by coloration with alizarin red for calcium deposits visualization;

(b) Osteogenic differentiation was detected by coloration analysis of alkaline phosphatase;

(c) Chondrogenic differentiation was detected by coloration with Alcian Blue for visualization of glycosaminoglycan reach matrix products;

(d) Adipogenic differentiation was detected by coloration with oil red for visualization of intracellular lipidic vacuoles.

### TERMS AND DEFINITIONS

Different terms related to the objects of present invention are used above and in description and claims of the invention. Unless otherwise specified, all technical and scientific terms used herein have the same meaning which is understood by professionals in the given field. References to methods used in description of this invention are related to well known methods including changes to these methods and substitution thereof with equivalent ones well known to professionals.

In description of this invention, the terms “includes” and “including” are interpreted as meaning “includes among other things”. The said terms are not intended to be understood as “only consists of”.

In this context, the term “medium” is related to any medium intended for cultivation and/or crystallization or recrystallization.

In description of this invention the terms “three-dimensional (3D) material based on calcium phosphate” or “framework” or “scaffold” are related to bone tissue engineering. These materials shall be porous for favorable intergrowth of biological bone during bone defect restoration.

### Embodiment of Invention

α-TCP Particles Fabrication

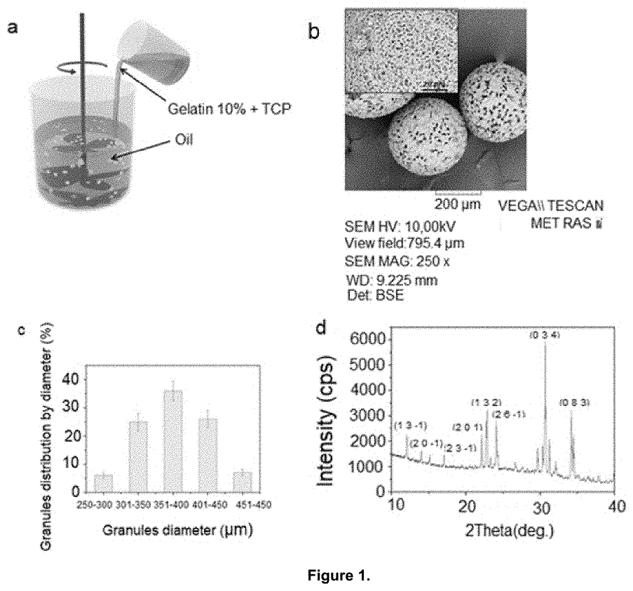

SEM micrographs of primary α-TCP ceramic particles intended for fabrication of 3D scaffold based on CP in magnetic field are shown in FIG. 1. Average size of α-TCP particles was about 250 to 500 μm. Resulting α-TCP crystals had globular morphology with maximum diameter of up to several microns.

Computer-Aided Simulation of Magnetic Field and Particles Assembly

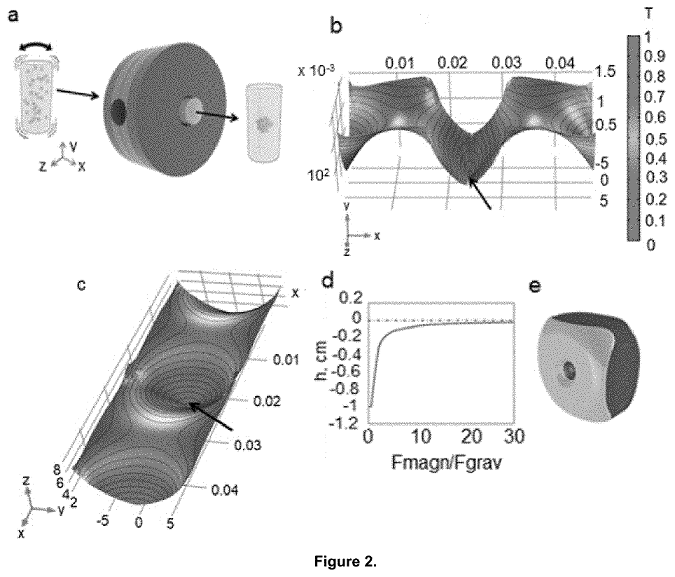

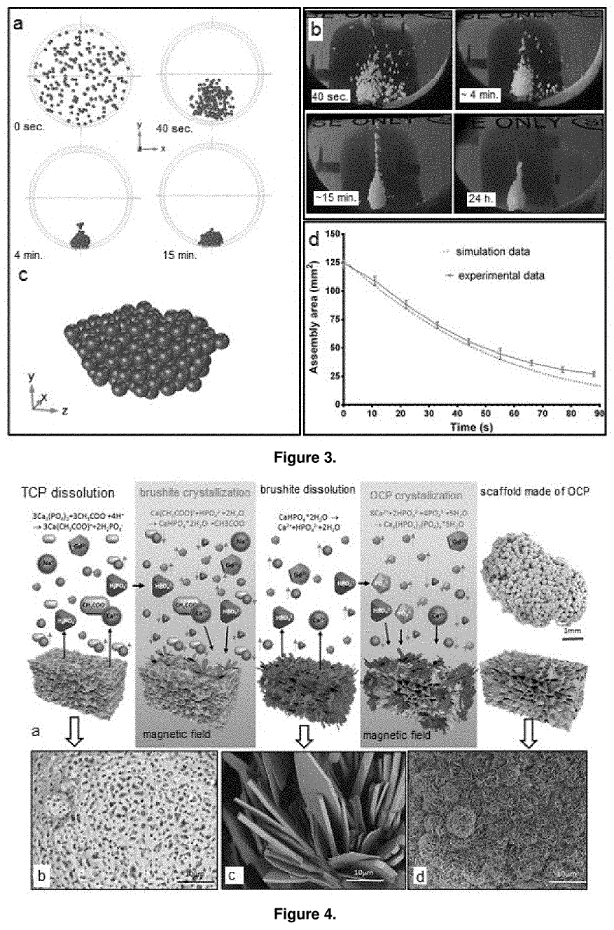

Authors of the present invention used magnetic device for assembly (FIG. 2a) which creates non-homogeneous magnetic field in working area. Magnetic field used as a temporary scaffold in this study was simulated using COMSOL software. Magnetic induction values density in vertical and horizontal sections of working area is shown in 3D model graph (FIG. 2b, c). Magnetic device provides particles levitation at different heights depending on the ratio between magnetophoretic and gravity forces which is in turn regulated by concentration of paramagnetic salt in the medium (FIG. 2d). Computer-aided simulation was used to define optimal concentration of paramagnetic salt in buffer solution and required magnetic field configuration which is necessary to ensure levitation assembly. Simulation also allows assessment of scaffold shape, assembly time and levitation height (FIG. 3). Scaffold assembly height depends on concentration of paramagnetic salt in the medium, and if it is high enough, generated magnetophoretic force can overcome gravitation and lead to granules flotation. Due to variable magnetic field gradient in vertical direction interaction between magnetic and gravitation forces acting on the scaffold also depends on assembly height. Peak value of magnetophoretic force is observed in the center of the field inside the magnets, but due to gravitation force the scaffold can be placed in the lower magnetic field area. Therefore, the value of magnetophoretic force shall exceed gravity force by several times to hold the scaffold at some height over the bottom. Increase of this ratio will lead to increase of scaffold assembly height. It is worth noting that computer-aided simulation requires that liquid and particles' characteristics are taken into account. While density can be determined quite easily, CP permeability was not known a priori. Medium viscosity in given conditions was evaluated by experiment: granule movement was recorded to determine its velocity and viscosity was calculated according to Stokes law. In the present study, velocity was very low so Reynolds number was lower than 1. In such case, specific resistance of liquid was described quite precisely by Stokes law. Using all other known parameters of CP and medium, magnetic permeability of particles can be calculated by comparison between assembly time in pilot experiment and computer-aided simulation. It was equal to μₚ=0.99999 which is very close to magnetic permeability of bone.

Scaffolds (Frameworks) Fabrication Using Magnetic Levitation Assembly

While computer-aided simulation predicted spherical shape of final scaffold (FIG. 3a) elongation of vertical shape was visualized after assembly (FIG. 3b). Such shape may be explained by the change of particles density due to some differences in porosity. Also worth noting is that after 20 hours of levitation in buffer No. 1 particles' sedimentation on the scaffold began due to pores filling. Visible area of CP conglomerate was measured to compare simulated and experimental assembly processes. Results are shown in FIG. 3c. Experimentally measured area decreased until particles were packed compactly while final scaffold area was dependent on particles number. Computer-aided assembly simulation was performed for a small number of particles, therefore, the final scaffold size was comparable to granules size. Despite of the difference in final scaffold size between experimental data and computer-aided simulation, intermediate time dependence for conglomerate area was similar in both cases. Therefore, particles convergence rate does not depend on particles number and consistently decreases with time.

In order to ease recrystallization during assembly the scaffold levitated in magnetic field during 40 hours at room temperature. Process of α-TCP conversion to OCP is shown in FIG. 4a. At the end of step 1 initial structure of α-TCP (FIG. 4b and FIG. 1) recrystallized to dicalcium phosphate dihydrate (brushite, DCPD) (FIG. 4c and FIG. 6). α-TCP on the surface of 3D scaffold began to dissolve in buffer No. 1 due to interaction of the surface with sodium acetate dissociation products accompanied by emission of Ca²⁺, H₂PO₄⁻ (prevails in solution) and HPO₄²⁻ in solution. When concentration of these ions in interphase reached its defined critical value, crystallization occurred. Saturation level increased due to application of non-homogeneous magnetic field since concentration of anions and cations grew faster due to magnetic sedimentation effect. Increased concentration of ions in solution led to generation of DCPD crystals on the surface of α-TCP with subsequent growth thereof. DCPD generation reaction was as follows:

2Ca₃(PO₄)₂₊2NaH₂PO₄+2CH₃COOH+12H₂O=6CaHPO₄.2H₂O+2CH₃COONa

At the end of step 2 the whole structure was fully converted to OCP (FIG. 4d and FIG. 6). On the first hand, DCPD crystals on the surface of 3D scaffold obtained as a result of step 1 began to dissolve in buffer No. 2. Dissolution took place due to interaction of DCPD crystals with acetic acid and sodium hydroxide dissociation products which contribute to the increase of Ca²⁺ cations and H₂PO₄⁻ and HPO₄²⁻ anions concentration in interphase. Similar to step 1, magnetic sedimentation contributed to the increase of ions concentration in interphase which led to sedimentation of OCP and generation of OCP crystals on DCPD surface. OCP generation reaction may be expressed as follows:

8CaHPO₄.2H₂O+8NaOH+4CH₃COOH→Ca₈(HPO₄)₂(PO₄)₄.5H₂O+2Na₂HPO₄+4CH₃COONa+19H₂O

Gibbs energies were calculated based on standard activity of ions presented in solutions. ΔG of DCPD generation is equal to −7 kJ/mole, and ΔG of OCP generation is equal to −663 kJ/mole. It confirms the possibility of these reaction.

SEM image of 3D scaffold which was obtained in similar buffer conditions but without magnetic field application (considered as a reference sample) is shown in FIG. 7. OCP crystals generation was not observed on reference sample surface which may be explained by the absence of magnetic field. Indeed, levitation state in magnetic field contributes to OCP generation in a quite short time, in particular, within 40 hours.

Biocompatibility of 3D Scaffolds

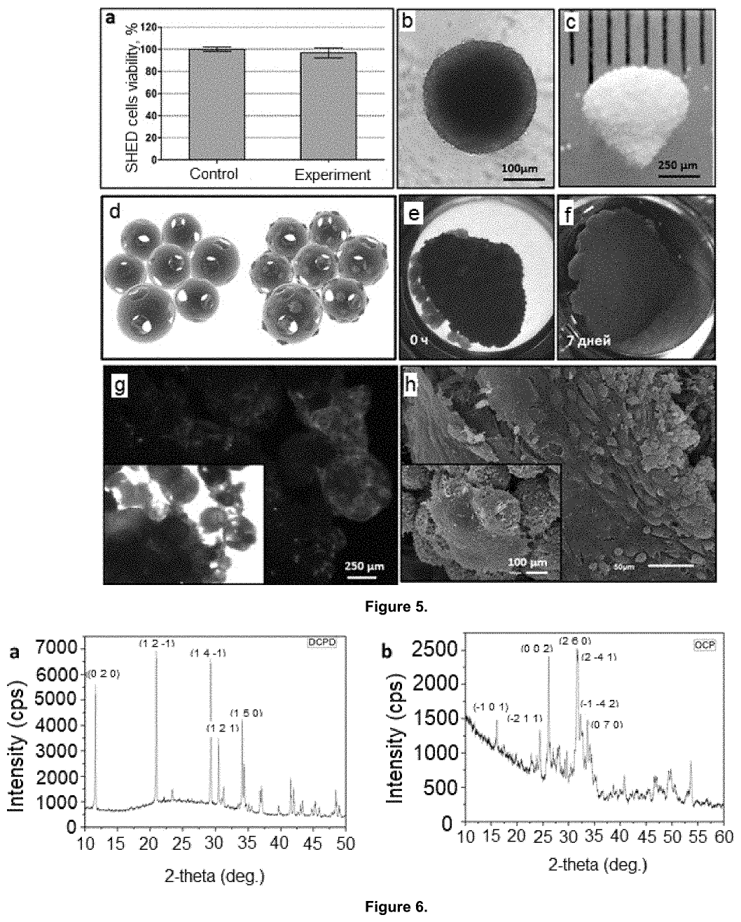

In order to evaluate cytotoxicity of 3D scaffolds extract-based cytotoxicity analysis was used which evaluates cytotoxicity of any secondary products extracted from material. FIG. 5a shows that SHED cells in culture medium preliminarily incubated with 3D scaffold retain 97% of viability after 72 hours of incubation in the presence of extracts.

Surface properties of 3D scaffold in relation to cellular colonization were evaluated using fluorescent microscopy and SEM. Schematic image of SHED cells migration from tissue spheroids and their adherence to the surface of 3D scaffold is shown in FIG. 4b. One-day tissue spheroids made of SHED cells (FIG. 5c) were used which then were incubated in close contact with 3D scaffold (FIG. 5d) over 7 days (FIG. 5e, f). It can be seen that tissue spheroids adhere to 3D scaffold and distribute evenly on its surface after 7 days of cultivation (FIG. 5f to g). It indicates non-toxicity and high biocompatibility of 3D scaffold based on OCP obtained using the method in accordance with the present invention.

### Examples of Embodiment of the Invention

It should be understood, that these and all examples given in application materials are not limiting and are given only for illustration of the present invention.

Tools and Methods

Chemicals and Reagents

Dulbecco's Minimal Essential Medium (DMEM, cat. No.: 12491-015), Fetal Bovine Serum (FBS, cat. No.: 16000-044), antibiotic/antimycotic agent (cat. No.: 15240-062), trypsin/EDTA (cat. No.: 25200-114) were received from Gibco (USA). EDTA (cat. No.: R080), L-glutamine (cat. No.: F032) was received from PANECO (Russia). High-purity calcium nitrate (cat. No.: 13477-34-4), ammonium carbonates (cat. No.: 506-87-6), diammonium phosphate (cat. No.: 7722-76-1), sodium acetate (cat. No.: 127-09-3), glutaraldehyde (cat. No.: G5882) and resazurin sodium salt (cat. No.: R7017-5G) were received from Sigma-Aldrich (USA).

α-TCP powder was synthesized in aqueous medium by slow addition of diammonium phosphate solution ((NH₄)₂HPO₄) to calcium nitrate solution (Ca(NO₃)₂.4H₂O) containing ammonia solution (NH₄OH) with constant stirring. Mixture pH was about 7 with molar Ca/P ratio of 1.5/1. After full addition of reagents the suspension was filtered, dried at 80° C. and sintered at 900° C. during 2 hours.

In order to obtain α-TCP particles the method was used which is based on spheroidization of liquid drops under surface tension forces using a mixed suspension of α-TCP, binding agent (gelatin) and oil (FIG. 1). This method allows to obtain porous spherical granules with open pores generated during binding agent burning out. Spherical particles generated under surface tension forces were filtered on Buchner funnel, rinsed to remove oil, dried and then heated to 1300° C. α-TCP particles with average size of 250 to 500 μm were used for scaffolds (frameworks) assembly (FIG. 1). α-TCP particles composition was confirmed using X-ray phase analysis (FIG. 1).

Two solutions were prepared to obtain magnetic levitation assembly and recrystallization of α-TCP particles. One buffer solution (called buffer No. 1 herein) was prepared by dissolution of 1.5±0.1 M of sodium acetate and 0.0±0.01 M of phosphoric acid in water (pH 5.2±0.2). The second buffer solution (called buffer No. 2 herein) was prepared by dissolution of CH₃COONa (95.2 g) in 700 ml of distilled water (which corresponds to 1.66 mole I⁻¹) with pH 8.2±0.2. Buffer No. 1 and buffer No. 2 both contained 3M of Gd³⁺. In order to obtain concentration of 3M, 3 ml of 1M Gadovist (BAYER PHARMA AG, Germany) were lyophilized using Heto PowerDry LL3000 (Thermo Fisher Scientific, USA) and dissolved in 1 ml of buffer No. 1 and buffer No. 2.

The Magnetic Device

Fabrication process was performed in two steps—α-TCP particles assembly in magnetic levitation conditions and their subsequent recrystallization. 3D scaffolds have been assembled by levitation forming using non-homogeneous magnetic field in the presence of Gd³⁺ salts at room temperature (RT). Magnetic device (FIG. 2a) consists of magnets retaining system and 2 circular NdFeB (N52) magnets. Outer diameter of magnets is 85 mm; inner diameter is 18 mm; thickness (height) is 24 mm. Magnets were assembled in the way allowing their placement with analogous poles facing each other. Non-homogeneous magnetic field was created in axial opening of magnetic device (working area). Distribution of magnetic induction values density is vertical and horizontal sections of working area is shown in 3D model diagram (FIG. 2b, c). Dependence of the distance between levitation unit and magnetic field center on the ratio between magnetophoretic and gravitation forces acting on the particles is shown in FIG. 2d. Cylindrical opening was made in the center of construction perpendicular to the axis of magnetic rings in order to observe fabrication process using two digital CMOS cameras, light sources and lens system. Magnetic device also includes ferromagnetic shield used for magnetic field shielding. Magnetic device appearance is shown in FIG. 2e. Geometry of circular magnets and geometry of through opening is chosen according to desired construct shape.

In order to start fabrication process, transparent syringe of 2 ml in volume (SMF, Germany) was filled with buffer No. 1 containing α-TCP particles (ratio between particles weight and liquid weight was 1:400). After intensive shaking particles distributed evenly in the whole liquid volume in syringe. When this syringe was inserted in the opening of magnet system, α-TCP particles began to accumulate in the center of working area. As a results, particles gathered in three-dimensional levitating scaffold under the action of magnetic forces. Once α-TCP particles are gathered in the scaffold, recrystallization process begins. It can be subdivided in two steps. During the first step, α-TCP particles remained levitating in buffer No. 1 during 20 hours. During the second step, syringe with 3D scaffold in buffer No. 1 was withdrawn from the printer, buffer No. 1 in the syringe was replaced by buffer No. 2 and then the syringe was placed in magnetic device again. After that the scaffold was subjected to further recrystallization in levitation conditions during additional 20 hours. Principle of operation of experimental device involves creation of local microgravitation zone where all forces acting on objects are compensated. Magnetophoretic force appears only if magnetic field is not homogeneous. It causes particles movement from areas where magnetic field is strong. Magnetophoretic force is applicable for neutrally charged particles whose relative permeability differs from that of background liquid. In such case, effective magnetophoretic force F acting on the object in non-homogeneous magnetic field will be defined as follows:

F=2πr³μ₀μ_{f}K∇(H²),

where H is magnetic field, μᵣ is relative liquid permeability, μₚ is relative particle(s) permeability, μ₀ is magnetic constant and K is defined as:

$$K = \frac{{μ}_{p} - {μ}_{f}}{{μ}_{p} + 2 {μ}_{f}} .$$

In this experiment relative permeability of liquid and particles is very close to 1, therefore, magnetophoretic force acting on particles is approximately linear in relation to the difference between them. Since μ1 for paramagnetic materials and μ<1 for diamagnetic materials, the difference μₚ−μᵣ refines magnetic force direction. As a result, objects will be ejected to the area with the lowest field density (magnetic trap) under the action of magnetophoretic force. In Earth gravitation conditions, equilibration of objects occurs at a certain distance from local minimum of magnetic field.

Computer-Aided Simulation of Magnetic Field and Particles Assembly

Simulation of three-dimensional non-homogeneous static magnetic field in paramagnetic medium made of two permanent magnets was performed using COMSOL Multiphysics modeling software by finite elements method. Characteristics used during magnetic field simulation were as follows: relative permeability of paramagnetic medium was μᵣ=1.00027; and magnet N38 made of NdFeB class material (VG=1.21 TL) was used. Magnetic field was calculated and then inserted in field particles trajectory equation. Intermediate calculation of particles trajectories was performed using COMSOL “Particles monitoring module”. The following forces were taken into account during calculation: magnetophoretic force based on the difference between magnetic permeability of particle and medium, resistance force affecting assembly time, particle-to-particle interaction elastic force and gravity force. Due to low particles' velocities Stokes resistance law was used to describe viscous resistance. Physical characteristics of particles were similar to those of CP particles: diameter was equal to 0.5 mm, density was taken equal to 2,000 kg m⁻³, particles' shape was supposed to be spherical, and total number of simulated particles was 400. Notwithstanding that solid CP density is equal to 2,800 kg m⁻³, particles contained air bubbles which decreased effective density. Specific properties of paramagnetic liquid were defined by experiment and had the following values: density was equal to 1,550 kg m⁻³, dynamic viscosity was equal to 0.01 Pa·s. Calculated particle velocities corresponded closely to experimental data.

Material Characteristics

Phase composition was analyzed using usual X-ray diffraction method (XRD) [Shimadzu XRD-6000 (Japan), Ni-filtered target CuKα1, λ=1,54183 Å]. Samples were scanned at 2θ=3°−60° with pitch of 0.02° during preset time of 5 seconds.

Scanning electron microscopy (SEM) apparatus (Tescan Vega II, Czech Republic) operating in secondary and backward scattering of electrons was used to research materials.

3D scaffolds based on CP with SHED cells were fixed with 2.% v/v glutaraldehyde PBS, dewatered in ethanol series and then dried in drier with critical point (HCP-2, Hitachi Koki Co. Ltd., Japan), and observation was performed using JSM-6510 LV microscope (JEOL, Japan).

All samples were coated with 25 nm thick gold layer by spraying using ion coating device (IB-3, EIKO, Japan) prior to visualization in order to add electrical conductivity to surfaces.

Cell Culture In this study, primary MSC adhesive cultures from primary teeth pulp collected after normal peeling-off—SHED (stem cells of peeled-off human primary teeth) were used (FIG. 8). Human primary teeth were collected from three healthy children (5 to 8 years old) after their normal peeling-off. Samples were stored in HBSS containing antibiotic/antimycotic agent (Gibco, USA) before delivery to laboratory over 24 hours. Pulp tissue was mechanically extracted from the crown, disintegrated and overcured in 0.1% type I collagenase solution in HBSS (Gibco, USA) during 60 minutes at 37° C. SHED cells (stem cells of peeled-off human primary teeth) were collected by centrifugation and suspended in growth medium (DMEM/F12 with addition of 10% of embryonal bovine serum and 100 units per ml of penicillin/streptomycin (Gibco, USA)). Cells were reproduced in 75 cm² flasks in standard conditions (37° C., humidified air containing 5% of CO₂). Cells were separated for passaging by incubation with 0.25% v/v trypsin/EDTA solution (Paneco, Russia) during 5 minutes at 37° C., reseeded in growth medium and subcultured in 1:3 ratio. Cells were free from mycoplasmal contamination which is confirmed by staining protocol using Hoechst 33258 (Sigma, Cat. No: 861405).

Primary SHED culture was analyzed by immunophenotyping method using flow cytometry for presence and absence of certain markers as recommended by International Society for Cellular Therapy (ISCT) [20]. It was found that cells were strongly positive (over 95%) for CD29, CD44, CD49b, CD73 and CD90 and negative (less than 2% positive) for CD34, CD45 and HLA-DR.

Authors of the present invention also checked cell cultures for multipotent ability for differentiation in osteogenetic, adipogenic and chondrogenic lines since the ability for differentiation in three different lines is another obligatory ISCT criterion for MSC (FIG. 9). Results shown that multipotent mesenchymal stromal cells were prevailing cells in our SHED cultures.

Tissue Spheroids Generation Using Corning Spheroidal Microplates

Tissue spheroids were generated using Corning spheroid microplates (Corning, cat. No.: 4520) according to manufacturer's protocol. Cells in monolayer with 95% fusion were rinsed with EDTA solution, gathered from substrate using 0.25% trypsin/0.53 mM EDTA and then resuspended in cell culture medium. Cells concentration was 2.7×10⁵ per milliliter. 100 μl of cell substrate were added in wells of Corning spheroidal microplates. Corning spheroidal were incubated at 37° C. in humid atmosphere with 5% of CO₂ over 24 hours.

Cytotoxicity Assay of 3D Scaffold Based on Calcium Phosphate

Extract was tested to evaluate cytotoxicity. The extract had the form of 3D scaffolds based on CP saturated in culture medium at 37° C. over 4 days. SHED cells were seeded in 96-well cultural plate in concentration of 1×10⁴ cells per well. Each well contained 100 μl of cell-rich fluid and the plate was incubated during 24 hours at 37° C. in humidified atmosphere with 5% of CO₂ to obtain the culture in front of monolayer. Then 200 μl of culture medium preliminarily incubated with 3D scaffold was inserted in experimental wells and 200 μl of fresh culture medium were inserted in reference wells. The plate was incubated during additional 72 hours at 37° C. in humidified atmosphere with 5% of CO₂, then 200 μl of supernatant were aspirated and 10 μl of 0.02% resazurin solution in culture medium were added to each plate well. Plate was returned to CO₂ incubator for 6 hours and then fluorescence was recorded at excitation wavelength of 555 nm with emission recorded at 580 nm using multibeam plate reader VICTOR X3 (Perkin Elmer, USA). Wells contained cell culture medium without any cells and were used for background signal evaluation.

Biocompatibility of 3D Scaffolds

Biocompatibility of 3D scaffolds was studied in relation to SHED tissue spheroids using fluorescent microscopy (NIKON SMZ18, USA) and SEM (JSM-6510 LV, JEOL, Japan). One-day tissue spheroids made of SHED cells were incubated in close contact with 3D scaffold based on CP during 7 days. Cells viability was controlled using a live/dead assay staining kit (Sigma-Aldrich, USA) according to manufacturer's protocol. This analysis was used to determine visually whether cells in tissue spheroids remain viable after cultivation using 3D scaffold based on CP. After 7 days of cultivation 3D scaffold with one-day tissue spheroids made of SHED cells was incubated with solution containing calcein-AM and propidium iodide (PI) at 37° C. during 30 minutes. After rinsing with PBS tissue spheroids were visualized using fluorescent microscopy (NIKON SMZ18, USA).

Data Analysis

Statistical data were analyzed using GraphPad Prism software (GraphPad Software, Inc., La Jolla, Calif.) and presented as average value±S.E.M. Analysis of variance (ANOVA) was used to determine significant differences between average values of three groups with P<0.0001.

Notwithstanding that the invention is described with reference to embodiments disclosed, it should be obvious for professionals in the given field that specific experiments detailed herein are only given for illustration of the present invention and they should not be considered as limiting the scope of invention in any way. It should be understood that realization of different modifications is possible without departing from the spirit and scope of the present invention.

List of literature sources included in this disclosure of the invention as references:
1. Giraud-Guille M.-M., Belamie E., Mosser G. Organic and mineral networks in carapaces, bones and biomimetic materials//Comptes Rendus Palevol. Elsevier Masson, 2004. Vol. 3, No 6-7. P. 503-513.
2. Suba Z. et al. [Quantitative and qualitative comparison of the maxillary bone regeneration after beta-tricalcium phosphate and autogenous bone implantation].//Fogorv. Sz. 2006. Vol. 99, No P. 21-28.
3. Horowitz R. A. et al. Clinical evaluation alveolar ridge preservation with a beta-tricalcium phosphate socket graft.//Compend. Contin. Educ. Dent. Vol. 30, No 9. P. 588-590, 592, 594 passim; quiz 604, 606.
4. Stavropoulos A. et al. Clinical and histologic evaluation of granular Beta-tricalcium phosphate for the treatment of human intrabony periodontal defects: a report on five cases.//J. Periodontol. 2010. Vol. 81, No 2. P. 325-334.
5. Raksujarit A. et al. Processing and properties of nanoporous hydroxyapatite ceramics//Mater. Des. 2010. Vol. 31, No 4. P. 1658-1660.
6. Elliott J. C. (James C. Structure and chemistry of the apatites and other calcium orthophosphates. 387 p.
7. Komlev V. S. et al. 3D Printing of Octacalcium Phosphate Bone Substitutes.//Front. Bioeng. Biotechnol. 2015. Vol. 3. P. 81.
8. Guo D., Xu K., Han Y. The in situ synthesis of biphasic calcium phosphate scaffolds with controllable compositions, structures, and adjustable properties.//J. Biomed. Mater. Res. A. 2009. Vol. 88, No 1. P. 43-52.
9. Schumacher M. et al. Indirect rapid prototyping of biphasic calcium phosphate scaffolds as bone substitutes: influence of phase composition, macroporosity and pore geometry on mechanical properties.//J. Mater. Sci. Mater. Med. 2010. Vol. 21, No 12. P. 3119-3127.
10. Detsch R. et al. In vitro: osteoclastic activity studies on surfaces of 3D printed calcium phosphate scaffolds.//J. Biomater. Appl. 2011. Vol. 26, No 3. P. 359-380.
11. Rath S. N. et al. Osteoinduction and survival of osteoblasts and bone-marrow stromal cells in 3D biphasic calcium phosphate scaffolds under static and dynamic culture conditions.//J. Cell. Mol. Med. 2012. Vol. 16, No 10. P. 2350-2361.
12. Melchels F. P. W., Feijen J., Grijpma D. W. A review on stereolithography and its applications in biomedical engineering/Biomaterials. 2010. Vol. 31, No 24. P. 6121-6130.
13. Negro A., Cherbuin T., Lutolf M. P. 3D Inkjet Printing of Complex, Cell-Laden Hydrogel Structures//Sci. Rep. 2018. Vol. 8, No 1. P. 17099.
14. Herberg S. et al. Inkjet-based biopatterning of SDF-1β augments BMP-2-induced repair of critical size calvarial bone defects in mice//Bone. 2014. Vol. 67. P. 95-103.
15. Mirica K. A. et al. Using Magnetic Levitation for Three Dimensional Self-Assembly//Adv. Mater. 2011. Vol. 23, No 36. P. 4134-4140.
16. Tasoglu S. et al. Magnetic Levitational Assembly for Living Material Fabrication.//Adv. Healthc. Mater. 2015. Vol. 4, No 10. P. 1469-1476, 1422.
17. Tocchio A. et al. Magnetically Guided Self-Assembly and Coding of 3D Living Architectures//Adv. Mater. 2018. Vol. 30, No 4. P. 1705034.
18. Durmus N. G. et al. Magnetic levitation of single cells//Proc. Natl. Acad. Sci. 2015. Vol. 112, No 28. P. E3661-E3668.
19. Parfenov V. A. et al. Scaffold-free, label-free and nozzle-free biofabrication technology using magnetic levitational assembly.//Biofabrication. 2018. Vol. 10, No 3. P. 034104.
20. Prockop D. J. et al. Minimal criteria for defining multipotent mesenchymal stromal cells. The International Society for Cellular Therapy position statement/Cytotherapy. 2006. Vol. 8, No 4. P. 315-317.

1. A method of fabrication of 3D material based on octacalcium phosphate by magnetic levitation assembly in non-homogeneous magnetic field including the following steps:
1) α-tricalcium phosphate particles assembly in 3D structures,
2) recrystallization of obtained structures.

2. The method according to the claim 1, wherein magnetic levitation assembly is performed in the central area on non-homogeneous magnetic field with lowest field density parameters from 3D material randomly distributed within fabrication chamber volume.

3. The method according to the claim 1, wherein non-homogeneous magnetic field is created using magnetic system consisting of at least two circular neodymium magnets with analogous poles facing each other.

4. The method according to the claim 1, wherein non-homogeneous magnetic field is created using Bitter magnets.

5. The method according to the claim 1, wherein the size of α-tricalcium phosphate particles is 250 to 500 μm.

6. The method according to the claim 1, wherein α-tricalcium phosphate particles assembly is performed by levitation forming method using non-homogeneous magnetic field in paramagnetic medium.

7. The method according to the claim 6, wherein paramagnetic medium with Gd³⁺ gadolinium salts is used.

8. The method according to the claim 1, wherein α-tricalcium phosphate particles assembly is performed at temperature of 0 to 80° C.

9. The method according to the claim 8, wherein α-tricalcium phosphate particles assembly is performed at room temperature.

10. The method according to the claim 1, wherein α-tricalcium phosphate particles are placed in magnetic field in buffer No. 1 with mass ratio of α-tricalcium phosphate particles to buffer No. 1 equal to 1:100 to 1:400, where buffer No. 1 is an aqueous solution of 1.5±0.1 M of sodium acetate and 1.0±0.01 M of phosphoric acid with pH 5.2±0.2 and also contains 3M of Gd³⁺.

11. The method according to the claim 10, wherein mass ratio of α-tricalcium phosphate particles to buffer No. 1 is 1:400.

12. The method according to the claim 1, wherein recrystallization of obtained structures is performed in two stages: in the first stage obtained 3D structures are held in magnetic field during 12 to 48 hours, then buffer No. 1 is replaced by buffer No. 2 and held in magnetic field during more 12 to 48 hours, where buffer No. 2 represents an aqueous solution of 1.5±0.1 M of sodium acetate with pH 8.2±0.2 and also contains 3M of Gd³±.

13. The method according to the claim 12, wherein obtained 3D structures are held in magnetic field during 20 hours in the first stage.

14. The method according to the claim 12, wherein 3D structures are held in magnetic field during 20 hours after buffer No. 1 replacement by buffer No. 2.

15. The method according to the claim 10, wherein α-tricalcium phosphate particles are placed in magnetic field in buffer No. 1 in transparent syringe and preliminarily shaken.

16. Three-dimensional material based on octacalcium phosphate for restoration and regeneration of bone defect fabricated using the method according to claim 1.
